(19) United States
(12) Patent Application Publication
(10) Pub. No.: US 2025/0308097 A1
(43) Pub. Date: Oct. 2, 2025

(54) INFORMATION PROCESSING SYSTEM, INFORMATION PROCESSING APPARATUS, INFORMATION PROCESSING METHOD, AND PROGRAM

(71) Applicant: RIGAKU CORPORATION, Tokyo (JP)

(72) Inventor: Takumi OTA, Tokyo (JP)

(73) Assignee: RIGAKU CORPORATION, Tokyo (JP)

(21) Appl. No.: 19/089,981

(22) Filed: Mar. 25, 2025

(30) Foreign Application Priority Data
Mar. 27, 2024 (JP) 2024-050720

(51) Int. Cl. G06T 11/00

(52) U.S. Cl. CPC G06T 11/005; G06T 2210/41

(57) ABSTRACT

According to an aspect of the present disclosure, an information processing system for processing a plurality of projection images taken by a CT device is provided. The information processing system includes a feature value acquisition unit, a spectral decomposition unit, and a synchronization processing unit. The feature value acquisition unit is configured to acquire waveform data, the waveform data being indicated as feature values obtained from each of the plurality of projection images. The spectral decomposition unit is configured to acquire decomposed waveform data by applying a predetermined analytical method to the waveform data, the decomposed waveform data being data of a waveform obtained by decomposing the waveform data. The synchronization processing unit is configured to determine a projection image to be used for reconstruction from among the plurality of projection images based on the waveform indicated by the decomposed waveform data.

### CROSS REFERENCE TO RELATED APPLICATION

The present application claims priority under 35 U.S.C. § 119 to Japanese Patent Application No. 2024-050720, filed Mar. 27, 2024, the contents of which are incorporated herein by reference in their entirety.

### BACKGROUND

The present disclosure relates to an information processing system, an information processing apparatus, an information processing method, and a program.

### RELATED ART

JP 6348865 B discloses a technique for easily extracting only accurate periodic motion of one of the heartbeat or respiratory rates by removing noise caused by the other.

However, the invention described in JP 6348865 B requires that a certain periodicity be assumed in the movement of a subject when processing images.

In view of the above circumstances, the present disclosure provides a technique that can reconstruct an image or that assists in a reconstruction processing, without assuming a certain periodicity in the movement of a subject.

### Means for Solving Problems

According to an aspect of the present disclosure, an information processing system for processing a plurality of projection images taken by a CT device is provided. The information processing system, including: circuitry configured to: acquire waveform data, and the waveform data being indicated as feature values obtained from each of the plurality of projection images taken by a CT device; acquire decomposed waveform data by applying a predetermined analytical method to the waveform data, the decomposed waveform data being data of a waveform obtained by decomposing the waveform data; and determine a projection image to be used for reconstruction from among the plurality of projection images based on the waveform indicated by the decomposed waveform data.

According to the present disclosure, it is possible to reconstruct an image or assist in a reconstruction processing without assuming a certain periodicity in the movement of a subject.

### BRIEF DESCRIPTION OF DRAWINGS

FIG. 1 shows an example of a system configuration and a hardware configuration of an information processing system 1.

FIG. 2 shows an example of functional units included in a processor 21.

FIG. 3 shows an example of activities executed by the information processing system 1.

FIG. 4 is a diagram for explaining that decomposed waveform data is acquired from waveform data.

FIG. 5 is a diagram for explaining a method of determining an exhalation area and an inhalation area.

FIG. 6 is a diagram for explaining a method of determining exhalation and inhalation of the lungs and a diastole and a systole of the heart.

FIG. 7 shows an example of the results of determining the exhalation and the inhalation of the lungs and the diastole and the systole of the heart by the method of JP 6348865 B.

FIG. 8 shows an example of a screen 4.

FIG. 9 shows a reconstructed image of the lungs when synchronization is not performed.

FIG. 10 shows a reconstructed image of the lungs when exhalation of the lungs is synchronized in the same ROI as in FIG. 9.

FIG. 11 shows a reconstructed image of the lungs when inhalation of the lungs is synchronized in the same ROI as in FIG. 9.

FIG. 12 shows a reconstructed image of the heart when synchronization is not performed.

FIG. 13 shows a reconstructed image of the heart when a diastole of the heart is synchronized in the same ROI as in FIG. 12.

FIG. 14 shows a reconstructed image of the heart when a systole of the heart is synchronized in the same ROI as in FIG. 12.

### DETAILED DESCRIPTION

### Embodiment

Hereinafter, an embodiment of the present disclosure will be described with reference to drawings. Various features described in the embodiment below can be combined with each other.

A program for realizing a software in the present embodiment may be provided as a non-transitory computer readable medium that can be read by a computer or may be provided for download from an external server or may be provided so that the program can be activated on an external computer to realize functions thereof on a client terminal (so-called cloud computing).

A term “unit” in the present embodiment may include, for example, a combination of hardware resources implemented as circuits in a broad sense and information processing of software that can be concretely realized by these hardware resources. Furthermore, various kinds of information are described in the present embodiment, and such information may be represented by, for example, physical values of signal values representing voltage and current, high and low signal values as a set of binary bits consisting of 0 or 1, or quantum superposition (so-called qubits), and communication/calculation can be performed on a circuit in a broad sense.

The circuit in a broad sense is a circuit realized by combining at least an appropriate number of a circuit, a circuitry, a processor, a memory, and the like. In other words, a circuit includes an application specific integrated circuit (ASIC), a programmable logic device (e.g., simple programmable logic device (SPLD), a complex programmable logic device (CPLD), field programmable gate array (FPGA)), and the like. The circuit also includes a serverless architecture that uses container-based services to run applications in an environment abstracted from the management of physical infrastructure.

### 1. System Configuration and Hardware Configuration of Information Processing System 1

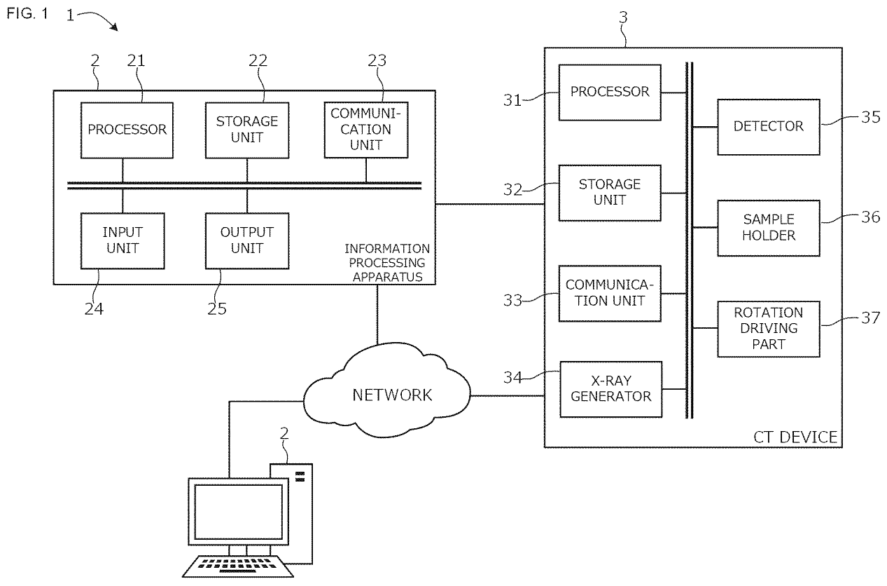

Firstly, a system configuration and a hardware configuration of an information processing system 1 of the present embodiment will be described with reference to FIG. 1. FIG. 1 shows an example of a system configuration and a hardware configuration of the information processing system 1.

### (Information Processing System 1)

The information processing system 1 shown in FIG. 1 is configured to process a plurality of projection images taken by a CT device 3. The information processing system 1 includes an information processing apparatus 2 and a computed tomography (CT) device 3. The information processing apparatus 2 and the CT device 3 are configured communicatively with each other via a communication cable or a network. This allows the information processing apparatus 2 and the CT device 3 to transmit or receive various information to each other. Here, a system exemplified by the information processing system 1 comprises one or more devices or components. Thus, even information processing apparatus 2 alone or CT device 3 alone can be included in the system exemplified by the information processing system 1. The information processing apparatus 2 and the CT device 3 are operated by a user who is, for example, a measurer.

### (Information Processing Apparatus 2)

The information processing apparatus 2 is a PC (Personal Computer). The information processing apparatus 2 may be a tablet-type computer, a smart phone, etc., instead of a PC. The information processing apparatus 2 is configured to process a plurality of projection images taken by the CT device 3. Specifically, for example, the information processing apparatus 2 is configured to be able to control arbitrary information processing to measurement data acquired from the CT device 3, control an X-ray generated from an X-ray generator 34, acquire a projection image detected by a detector 35, control movement of a sample holder 36, control a rotation driving part 37, and the like. The information processing apparatus 2 may be able to execute arbitrary information processing related to the CT device 3 as a result, and another information processing apparatus may be intervened between the information processing apparatus 2 and the CT device 3. As shown in FIG. 1, the information processing apparatus 2 includes a processor 21, a storage unit 22, a communication unit 23, an input unit 24 and an output unit 25, and these components are electrically connected via a communication bus inside the information processing apparatus 2. The information processing apparatus 2 executes processing according to the embodiment.

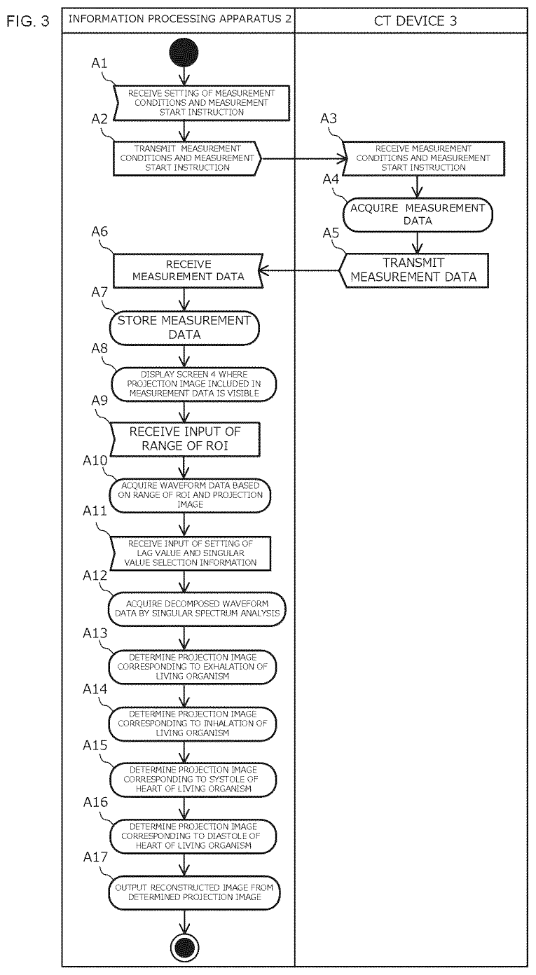

The processor 21 processes and controls overall operations related to the information processing apparatus 2. The processor 21 is, for example, a central processing unit (CPU). The information processing by a program stored in the storage unit 22 is specifically realized by the processor 21, which is an example of the hardware, thereby being executed as each functional unit included in the processor 21. Each functional unit included in the processor 21 realizes, for example, the processing shown in FIG. 3, which will be described later. The processor 21 is not limited to be a single and may be implemented with a plurality of processors 21 for each function. Also, a combination thereof may be applied as well.

The storage unit 22 stores various information defined by the above description. This may be implemented, for example, as a storage device such as a solid state drive (SSD) storing various programs related to the information processing apparatus 2 that are executed by the processor 21, or as a memory such as a random access memory (RAM) that stores temporarily necessary information (argument, sequence, etc.) pertaining to program operations. The storage unit 22 stores various programs and variables related to the information processing apparatus 2, which are executed by the processor 21, and data used by the processor 21 to execute the processing based on the programs. The storage unit 22 may be an example of a storage medium.

The communication unit 23 is preferably a wire communication means such as USB, IEEE1394, Thunderbolt (registered trademark), wired LAN network communication, etc., but may also include wireless LAN network communication, mobile communication such as LTE/3G/4G/5G, Bluetooth (registered trademark) communication, and the like as necessary. In other words, it is more preferable to implement the system as a set of these multiple communication means. That is, the information processing apparatus 2 may communicate various information from outside via the communication unit 23.

The input unit 24 may be included in a housing of the information processing apparatus 2, or may be externally attached. For example, the input unit 24 may be integrated with the output unit 25 and implemented as a touch panel. If the input unit 24 is implemented as a touch panel, a user can input by tap operations, swipe operations and the like. Needless to say, a switch button, a mouse, a keyboard or the like may be employed instead of a touch panel. That is, the input unit 24 receives input based on the operation performed by the user. The input is transferred as an instruction signal via the communication bus to the processor 21, and the processor 21 may execute predetermined control or calculation as necessary.

The output unit 25 can function as a display device of the information processing apparatus 2. The output unit 25 may be included in the housing of the information processing apparatus 2, or may be externally attached. The output unit 25 displays a screen of a graphical user interface (GUI) that can be operated by a user. This should be implemented, for example, by using different display devices such as a CRT display, a liquid crystal display, an organic EL display, a plasma display, or the like, depending on the type of the information processing apparatus 2.

### (CT Device 3)

The CT device 3 is a device capable of irradiating a sample with X-rays and acquiring a projection image of the sample from the amount of the transmitted X-rays. The CT device 3 may include a sample rotation-type CT device that rotates the sample holder 36, a gantry-type CT device that rotates the X-ray generator 34 and the detector 35 relative to the sample holder 36, and the like. However, the CT device 3 is not limited thereto. The CT device 3 includes a processor 31, a storage unit 32, a communication unit 33, the X-ray generator 34, the detector 35, the sample holder 36, and the rotation driving part 37, and these components are electrically connected via a communication bus inside the CT device 3. The CT device 3 executes the processing according to the embodiment. As to the processor 31, the storage unit 32 and the communication unit 33 of the CT device 3, the description of the processor 21, the storage unit 22 and the communication unit 23 of the information processing apparatus 2 is to be referred.

The X-ray generator 34 irradiates X-rays toward an area including the sample placed on the sample holder 36. Furthermore, the X-ray generator 34 may also be configured to irradiate X-rays consisting of characteristic X-rays such as CuKα and FeKα.

The detector 35 is configured to detect X-rays transmitted through a sample placed on the sample holder 36. The detected X-rays are analyzed as measurement data by the information processing apparatus 2. The measurement data is data obtained by the measurement using the CT device 3. The measurement data includes information indicating an angle at which the image was taken and information on the projection image corresponding to the angle. As the detector 35, a two-dimensional detector using a CCD, an imaging plate or the like may be used.

The sample holder 36 is configured to hold a sample stage. The sample holder 36 may be configured to move the sample stage in an arbitrary direction based on a movement instruction generated by the processor 21 or the processor 31. The sample stage is configured to allow a sample to be placed thereon.

The rotation driving part 37 is configured to rotate the sample holder 36 and/or the X-ray generator 34 and the detector 35. The rotation driving part 37 may be configured to include a mechanism capable of adjusting a magnification ratio of a projection image when the image is taken.

### 2. Functional Configuration of Processor 21 of Information Processing Apparatus 2

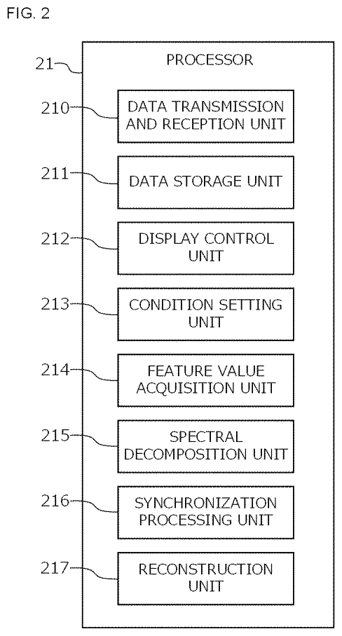

FIG. 2 shows an example of functional units included in the processor 21. As shown in FIG. 2, the processor 21 of the information processing apparatus 2, which is an example of the information processing system 1, includes a data transmission and reception unit 210, a data storage unit 211, a display control unit 212, a condition setting unit 213, a feature value acquisition unit 214, a spectral decomposition unit 215, a synchronization processing unit 216, and a reconstruction unit 217. As mentioned above, information processing by software stored in the storage unit 22 is specifically realized by the processor 21, which is an example of hardware, and thus can be executed as each functional unit (step) included in the processor 21. The processor 21 executes at least a feature value acquisition step, a spectrum decomposition step, a synchronization processing step, a condition setting step, and a display control step.

The data transmission and reception unit 210 receives or acquires various data inputs from the user via the input unit 24. The data transmission and reception unit 210 transmits various data to the CT device 3 via the communication unit 23. The data transmission and reception unit 210 accepts, receives or acquires various data from the CT device 3 via the communication unit 23.

The data storage unit 211 allows the storage unit 22 to store the data acquired from the CT device 3.

The display control unit 212 is configured to control the display information displayed on the output unit 25. The display information may be visual information itself, such as screen, image, icon, text, etc., generated in a visually recognizable manner for the user, or may be, for example, rendering information for displaying visual information such as screen, image, icon, text, etc. on various terminals.

The condition setting unit 213 receives settings of conditions such as ROI, lag value, singular value, etc.

The feature value acquisition unit 214 calculates a feature value from the projection image.

The spectral decomposition unit 215 acquires decomposed waveform data by applying a predetermined analytical method to the waveform data.

The synchronization processing unit 216 determines the projection image to be used for reconstruction from among a plurality of projection images.

The reconstruction unit 217 reconstructs an image of a sample from among the plurality of projection images.

Details of the data transmission and reception unit 210, the data storage unit 211, the display control unit 212, the condition setting unit 213, the feature value acquisition unit 214, the spectral decomposition unit 215, the synchronization processing unit 216, and the reconstruction unit 217 will be described later.

### 3. Flow of Action of Information Processing System 1

Next, a preferred example of information processing executed by the information processing system 1 of the present embodiment will be described. In this section, an example is described in which measurement data is acquired, waveform data and decomposed waveform data are further acquired from the measurement data, a projection image to be synchronized is determined by the decomposed waveform data, and the image is reconstructed, with reference to an activity diagram of FIG. 3. In the following embodiment, a description will be given assuming that singular spectrum analysis is applied as a predetermined analytical method. FIG. 3 shows an example of activities executed by the information processing system 1. The activities may include arbitrary exceptional processing. The exceptional processing includes interruption of the information processing and omission of each processing.

In the present embodiment, a plurality of projection images includes at least a part of the lungs or heart of a living organism as a subject. Living organisms may include humans and animals.

### (Activity A1)

First, the data transmission and reception unit 210 receives, via the input unit 24, a setting of the measurement conditions and an instruction to start measurement by the CT device 3 (hereinafter, referred to as a measurement start instruction) from the user. The measurement conditions include, for example, the number of images to be taken, scanning speed, exposure time, and a magnification ratio of the images to be taken.

### (Activity A2)

Then, the data transmission and reception unit 210 transmits the measurement conditions and the measurement start instruction to the CT device 3 via the communication unit 23.

### (Activity A3)

Then, the processor 31 of the CT device 3 receives the measurement conditions and the measurement start instruction from the information processing apparatus 2 via the communication unit 33.

### (Activity A4)

Then, the CT device 3 acquires measurement data based on the received measurement conditions.

### (Activity A5)

Then, the processor 31 of the CT device 3 transmits the measurement data to the information processing apparatus 2 via the communication unit 33.

### (Activity A6)

Then, the data transmission and reception unit 210 receives the measurement data from the CT device 3 via the communication unit 23.

### (Activity A7)

Then, the data storage unit 211 allows the storage unit 22 to store the acquired measurement data.

In a case where there is no need to acquire new measurement data (e.g., in a case where the reconstruction is executed by using the measurement data that has been already acquired), the information processing system 1 may omit the information processing of Activities A1 to A7.

### (Activity A8)

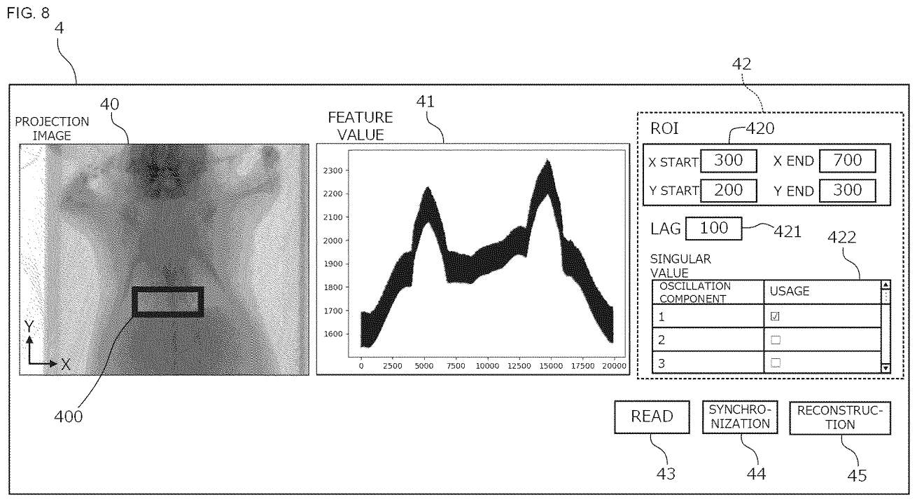

Next, the display control unit 212 causes the output unit 25 to display a screen 4 on which the user can visually recognize the projection image. The details of the screen 4 will be described later with reference to FIG. 8.

### (Activity A9)

Then, the condition setting unit 213 receives input for setting the range of the ROI on the screen 4 via the input unit 24.

The “ROI (Region of Interest)” is a partial area in a projection image from which feature values are acquired, and it is also referred to as a region of interest. In the embodiment, the ROI is a region that includes at least a part of the lungs and heart of a living organism.

### (Activity A10)

Then, the feature value acquisition unit 214 acquires waveform data based on the range of the ROI that has been received as input and the projection image. For example, the feature value acquisition unit 214 acquires feature values in the input range of the ROI for each of the plurality of projection images. The feature value acquisition unit 214 acquires waveform data by plotting the feature value of the ROI for each projection image on the vertical axis and the number of frames indicated by the projection images on the horizontal axis. The display control unit 212 further allows the waveform data to be displayed on the screen 4.

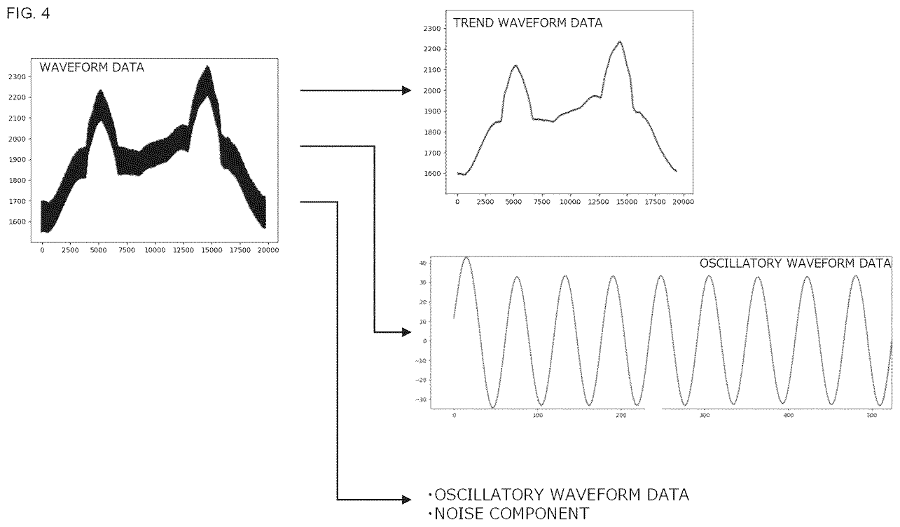

The “waveform data” refers to data indicated as feature values obtained from each of the plurality of projection images. In the embodiment, the waveform data is shown as a waveform as described below in FIG. 4.

The “feature value” is a value obtained by integrating the intensity in an image. In the present embodiment, the integrated value of the intensity in the ROI is used as the feature value. The feature values of the N projected images are x_1 to x_N, respectively, as shown in Equation 1. As the intensity, for example, a luminance value or the like may be used, but it is not limited thereto.

$$( {x}_{1} , {x}_{2} , {x}_{3} … , {x}_{N} ) \qquad [ Equation 1 ]$$

### (Activity A11)

Then, the condition setting unit 213 receives input of a setting of the lag value and singular value selection information from the user via the input unit 24 on the screen 4 on which the waveform data is visually recognizable to the user. According to such an aspect, it is possible to receive input for setting the conditions of the projection image to which the singular spectrum analysis is applied while the user visually recognizes the projection image, the waveform data, and the like. In addition, more appropriate decomposed waveform data can be acquired from the projection images based on the lag value arbitrarily set by the user.

The “lag value” is a value for specifying a Hankel matrix of the singular spectrum analysis from the feature values. For example, the number of rows in a Hankel matrix is represented by L, and the number of columns in a Hankel matrix is represented by K. L is equal to the lag value, and K is a value obtained by subtracting L and 1 from N which is the number of projection images, and can also be expressed as N−L−1.

The “singular value selection information” is information indicating which singular value is to be selected from among a plurality of singular values acquired from the matrix. In the present embodiment, the singular value selection information is information indicating which singular value is used to acquire decomposed waveform data from among a plurality of singular values acquired by the Hankel matrix.

### (Activity A12)

Next, the spectral decomposition unit 215 acquires decomposed waveform data by applying the singular spectrum analysis to the feature values of the waveform data based on the lag value.

More specifically, for example, the spectral decomposition unit 215 acquires a Hankel matrix shown in Equation 2 by applying a Hankel transform based on the feature value of Equation 1 and the lag value. The spectral decomposition unit 215 acquires a plurality of matrixes (X₁, X₂, . . . , Xᵣ) shown in Equation 3 by applying singular value decomposition to the acquired Hankel matrix. Each matrix is defined as shown in Equation 4 according to the singular value i. The spectral decomposition unit 215 acquires trend waveform data by applying an inverse Hankel transform to a matrix indicated by the maximum singular value (i=1) corresponding to the largest singular value among the singular values obtained by the singular spectrum analysis. The spectral decomposition unit 215 acquires oscillatory waveform data by applying an inverse Hankel transform to a matrix indicated by at least one or more singular values (corresponding to a singular value selected by the singular value selection information, for example, i=2) selected from the singular values other than the maximum singular value among the singular values obtained by the singular spectrum analysis. According to such an aspect, more appropriate decomposed waveform data can be acquired from the projection images based on the set lag value and singular value.

$$( \begin{matrix}{x}_{1} & {x}_{2} & {x}_{3} & … & {x}_{K} \\ {x}_{2} & {x}_{3} & {x}_{4} & … & {x}_{K + 1} \\ {x}_{3} & {x}_{4} & {x}_{5} & … & {x}_{K + 2} \\ … & ⋮ & ⋮ & ⋱ & ⋮ \\ {x}_{L} & {x}_{L + 1} & {x}_{L + 2} & … & {x}_{N}\end{matrix} ) \qquad [ Equation 2 ]$$

$$X = {X}_{1} + {X}_{2} + … + {X}_{r} \qquad [ Equation 3 ]$$

$${X}_{i} = {σ}_{i} {u}_{i} {v}_{i}^{T} \qquad [ Equation 4 ]$$

Here, the decomposed waveform data will be described with reference to FIG. 4. FIG. 4 is a diagram for explaining that decomposed waveform data is acquired from waveform data. As shown in FIG. 4, the waveform data is decomposed into a trend component (trend waveform data), oscillation components (oscillatory waveform data), noise components, etc. as decomposed waveform data. One or more oscillatory waveform data and noise components may be present. The intensity of all the decomposed waveform data is integrated to form waveform data.

The “decomposed waveform data” is data of a waveform obtained by decomposing waveform data. The decomposed waveform data includes at least trend waveform data, oscillatory waveform data, and noise components.

The “trend waveform data” is data of a waveform that has been decomposed as components indicating the trend of the waveform data. The trend waveform data reflects relatively large behaviors of, for example, the body, limbs, and the like of a living organism. The trend waveform data is also referred to as a first component.

The “oscillatory waveform data” is data of a waveform that has been decomposed as components indicating the oscillation of the waveform data. The oscillatory waveform data reflects behaviors of objects that move periodically, such as the lungs, heart, and the like of a living organism. The oscillatory waveform data is acquired from the second largest singular value and is also referred to as a second component.

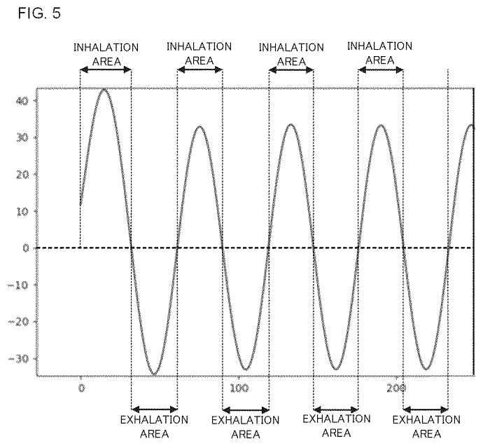

In Activities A13 to A16 described next, the synchronization processing unit 216 determines a projection image to be used for reconstruction from among a plurality of projection images based on a waveform indicated by at least one of the trend waveform data and the oscillatory waveform data. In explaining this information processing, a method of determining an exhalation area, an inhalation area, exhalation, inhalation, a diastole, and a systole by using the trend waveform data and the oscillatory waveform data will be described with reference to FIG. 5 to FIG. 7.

FIG. 5 is a diagram for explaining a method of determining an exhalation area and an inhalation area. FIG. 5 includes oscillatory waveform data. The oscillatory waveform data indicates oscillation components when the intensity indicated by the trend waveform data is set as a baseline (a predetermined value is zero).

The “exhalation area” is an area that can be considered to correspond to the exhalation of a living organism. In the present embodiment, the exhalation area is an area where the intensity of oscillation indicated by the oscillatory waveform data is lower than zero. In the example of FIG. 5, the exhalation area is an area where the intensity of the oscillatory waveform data is lower than zero (areas where the number of frames is around 40 to 70, around 90 to 110, around 150 to 180, etc.).

The “inhalation area” is an area that can be considered to correspond to the inhalation of a living organism. In the present embodiment, the inhalation area is an area where the intensity of oscillation is higher than zero. In the example of FIG. 5, the “inhalation area” is an area where the intensity of the oscillatory waveform data is higher than zero (areas where the number of frames is around 0 to 40, around 70 to 90, around 110 to 150, etc.). The point where the intensity is zero may be assigned to an exhalation area or an inhalation area.

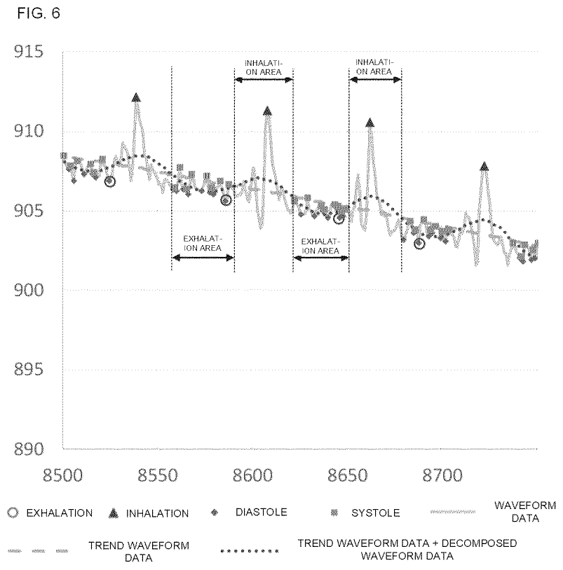

Next, a method of determining the exhalation and the inhalation of the lungs and the diastole and the systole of the heart from the exhalation area and the inhalation area determined by the method shown in FIG. 5 will be described. FIG. 6 is a diagram for explaining a method of determining the exhalation and inhalation of the lungs and the diastole and systole of the heart. In FIG. 6, the trend waveform data and the oscillatory waveform data reflecting the exhalation area and the inhalation area determined by the method shown in FIG. 5 are superimposed on the waveform data. In FIG. 6, the solid line indicates waveform data, the dashed line indicates trend waveform data, and the dotted line indicates data obtained by combining trend waveform data with oscillatory waveform data, respectively. In addition, in the trend waveform data of FIG. 6, disturbances in respiratory rates and heartbeats occur.

The exhalation is a part where the feature value becomes minimum at each of parts corresponding to a plurality of exhalation areas in the waveform data, for example, each part indicated by circles in FIG. 6.

The inhalation is a part where the feature value becomes maximum at each of parts corresponding to a plurality of inhalation areas in the waveform data, for example, each part indicated by triangles in FIG. 6.

The diastole is a part where the feature value becomes a local minimum value at each part corresponding to an exhalation area in the waveform data, for example, each part indicated by diamonds in FIG. 6.

The systole is a part where the feature value becomes a local maximum value at each part corresponding to an exhalation area in the waveform data, for example, each part indicated by squares in FIG. 6.

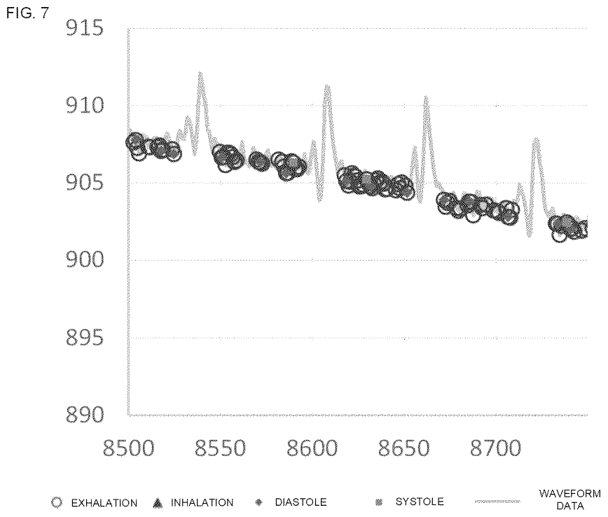

Next, a case where the method of JP 6348865 B is applied to the same trend waveform data as in FIG. 6 will be described with reference to FIG. 7. FIG. 7 shows an example of the results of determining the exhalation and the inhalation of the lungs and the diastole and the systole of the heart by the method of JP 6348865 B. In FIG. 7, circles indicate exhalation, triangles indicate inhalation, diamonds indicate diastoles, and squares indicate systoles, as in FIG. 6. In the method of JP 6348865 B, the determination of the exhalation and the inhalation of the lungs is not performed appropriately, and the number of diastolic and systolic data that can be obtained is small compared to the present disclosure. Thus, according to the present disclosure, even if the cycle of the respiratory rates or the heartbeats changes during measurement, the phrase (exhalation, inhalation, diastole, systole, etc.) can be determined more appropriately.

### (Activity A13)

The synchronization processing unit 216 determines, from the oscillation indicated by the decomposed waveform data, that an area where the intensity is lower than a predetermined value is an exhalation area. The synchronization processing unit 216 determines that each projection image in which the feature value becomes minimum at each of parts, in the waveform data, corresponding to a plurality of exhalation areas determined from the decomposed waveform data is a projection image corresponding to the exhalation of a living organism, and determines that this projection image is a projection image to be used for reconstructing the exhalation of the lungs.

### (Activity A14)

Then, the synchronization processing unit 216 determines, from the oscillation indicated by the decomposed waveform data, that an area where the intensity is higher than a predetermined value is an inhalation area. The synchronization processing unit 216 determines that each projection image in which the feature value becomes maximum at each of parts, in the waveform data, corresponding to a plurality of inhalation areas determined from the decomposed waveform data is a projection image corresponding to the inhalation of a living organism, and determines that this projection image is a projection image to be used for reconstructing the inhalation of the lungs.

### (Activity A15)

Then, the synchronization processing unit 216 determines that the projection image in which the feature value becomes a local maximum value at a part corresponding to the exhalation area in the waveform data is a projection image corresponding to the systole of the heart. The synchronization processing unit 216 determines a projection image to be used for reconstructing the systole of the heart from the projection images corresponding to the determined systole.

### (Activity A16)

Then, the synchronization processing unit 216 determines that the projection image in which the feature value becomes a local minimum value at a part corresponding to the exhalation area in the waveform data is a projection image corresponding to the diastole of the heart. The synchronization processing unit 216 determines a projection image to be used for reconstructing the diastole of the heart from the projection images corresponding to the determined diastole. The part corresponding to the exhalation area is a part where the intensity of the oscillatory waveform data is smaller than that of the trend waveform data, with the intensity of the trend waveform data being used as a reference.

When the information processing of the Activities A13 to A16 is completed, the display control unit 212 may display the decomposed waveform data that has been divided into areas shown in FIG. 5 and the waveform data with markers shown in FIG. 6 on a waveform display area 41 described below.

In a case where there is no need to synchronize exhalation and inhalation, the information processing system 1 may execute only the processing determined in the processing of Activities A13 and A14 and may omit the remaining processing. In a case where there is no need to synchronize systole and diastole, the information processing system 1 may omit processing of Activities A15 and A16.

According to Activities A13 to A16, it is possible to more appropriately determine a projection image to be used for reconstructing the lungs and the heart from the trend waveform data and the oscillatory waveform data obtained by singular spectrum analysis. In addition, even when elements such as noise and oscillation occur, a clearer image can be reconstructed from the projection image.

In response to an instruction from a user who has confirmed the result of synchronization, the information processing system 1 may proceed to information processing related to input of the setting of the range of ROI (Activity A9), input of the setting of the lag value and the singular value selection information (Activity A11), and acquisition and synchronization of the decomposed waveform data (Activity A12).

### (Activity A17)

Finally, the reconstruction unit 217 outputs an image that has been reconstructed (hereinafter, a reconstructed image) using a plurality of synchronized projection images for each of exhalation, inhalation, a systole and a diastole of a living organism.

The above is a description of the activities of the present embodiment. Next, the screen 4, the detailed description of which has been omitted will be described with reference to FIG. 8.

### (Screen 4)

FIG. 8 shows an example of the screen 4. The screen 4 is a screen on which information on the measurement data is displayed in a visually recognizable manner for the user. The screen 4 includes a projection image display area 40, a waveform display area 41, a condition reception area 42, a reading button 43, a synchronization execution button 44, and a reconstruction execution button 45.

The projection image display area 40 is an area where the acquired projection image is displayed. For setting the ROI, an X-axis and a Y-axis may be defined in the projection image in the projection image display area 40. The projection image display area 40 includes a ROI setting area 400. The ROI setting area 400 displays the area indicating the ROI in the projection image. The setting of the ROI range may be performed by any operation, such as drag-and-drop operation to the ROI setting area 400, changing the range to the ROI setting area 400 using a cursor, or inputting numerical values to the condition reception area 42, which will be described later. The condition setting unit 213 executes the setting of the range of the ROI as Activity A9 in FIG. 3.

The waveform display area 41 is an area where waveform data and/or decomposed waveform data are displayed. More specifically, for example, the waveform display area 41 may be an area in which the waveform data is displayed before the trend waveform data is acquired, and in which the waveform data and the trend waveform data are superimposed and displayed after the trend waveform data is acquired. The waveform display area 41 may be an area in which at least two of the waveform data, the trend waveform data, and the oscillatory waveform data are displayed side by side in a comparable manner. The condition reception area 42 is an area for receiving input of settings related to the singular spectrum analysis for the waveform data. The condition reception area 42 includes a ROI setting area 420, a lag setting area 421, and a singular value setting area 422.

The ROI setting area 420 is configured to set a ROI as a range in which waveform data is acquired from the projection image. For example, as shown in FIG. 8, the ROI setting area 420 may be configured to set a ROI by specifying coordinates from the X-axis and Y-axis defined in the projection image display area 40.

The lag setting area 421 is configured to set a lag value for defining the matrix of the singular spectrum analysis. For example, as shown in FIG. 8, the lag setting area 421 is configured to input “100” or the like as the lag value.

The singular value setting area 422 is configured to set which singular value of oscillation components is to be used to acquire oscillatory waveform data. For example, as shown in FIG. 8, the singular value setting area 422 is an area in which check boxes are displayed for determining which singular value of oscillation components is to be used in order of a larger singular value (1, 2, 3, etc.) associated with the oscillation components.

The reading button 43 is a button for reading the measurement data and displaying a projection image in the projection image display area 40. For example, the display control unit 212 executes Activity A8 in FIG. 3 in response to the pressing of the reading button 43.

The synchronization execution button 44 is a button for executing synchronization processing. In response to the pressing of the synchronization execution button 44, for example, the spectral decomposition unit 215 starts Activity A12 in FIG. 3, and then the synchronization processing unit 216 executes Activities A12 to A16 in FIG. 3.

The reconstruction execution button 45 is a button for executing reconstruction using a synchronized projection image. For example, the reconstruction unit 217 executes Activity A17 in FIG. 3 in response to the pressing of the reconstruction execution button 45.

### 4. Example

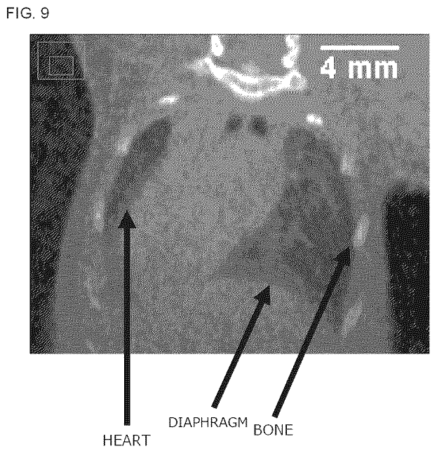

Next, an example of changes in reconstructed images before and after applying synchronization according to the present disclosure will be described with reference to FIG. 9 to FIG. 14. The subjects in the example are the lungs and heart of a mouse.

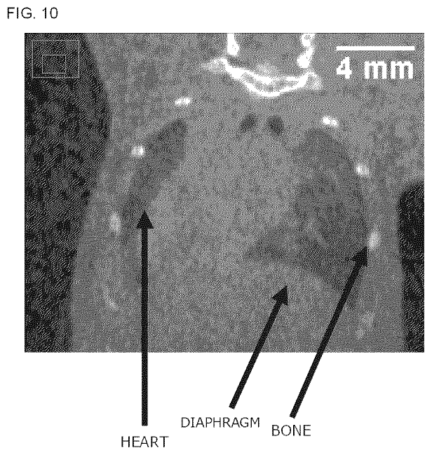

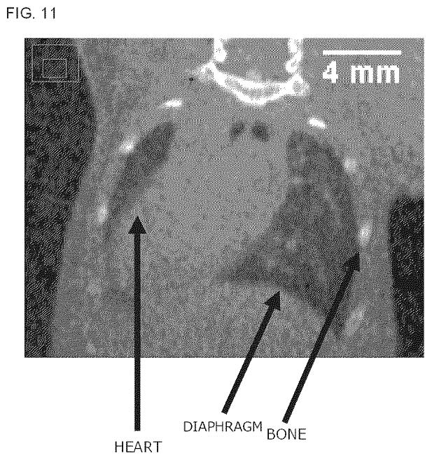

FIG. 9 shows a reconstructed image of the lungs when synchronization is not performed. FIG. 10 shows a reconstructed image of the lungs when the exhalation of the lungs is synchronized in the same ROI as in FIG. 9. FIG. 11 shows a reconstructed image of the lungs when the inhalation of the lungs is synchronized in the same ROI as in FIG. 9. In the reconstructed image before correction, the boundaries between the lungs and the space cannot be clearly recognized, especially in the areas indicated by the arrows in FIG. 9 (heart, diaphragm, and bones), and the contours of the lungs are blurred, resulting in a blurred reconstructed image. On the other hand, as shown in FIG. 10 and FIG. 11, in the reconstructed image after correction, the boundaries between the lungs and the space can be more clearly recognized at the areas indicated by the arrows similar to those in FIG. 9, so that it can be seen that the reconstructed image is clearer.

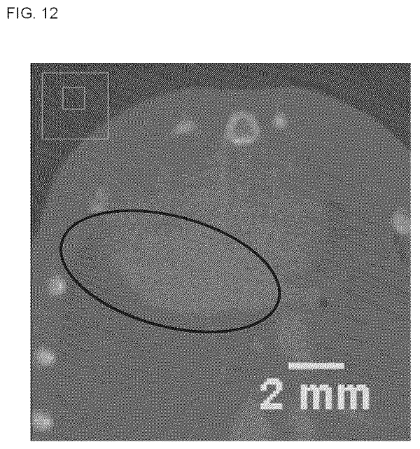

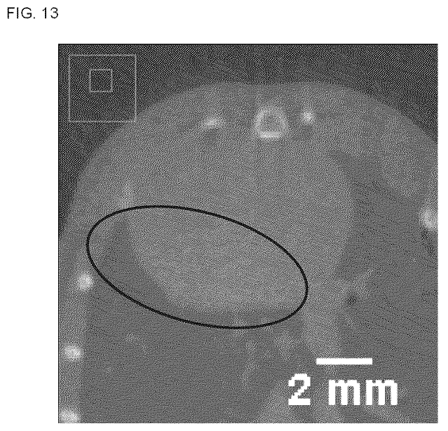

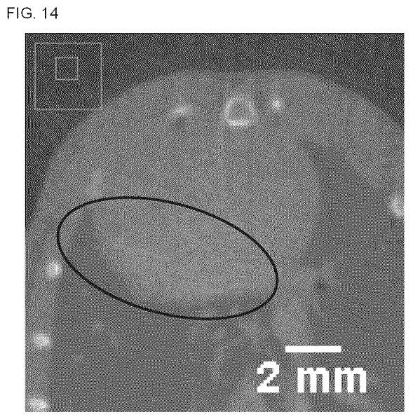

FIG. 12 shows a reconstructed image of the heart when synchronization is not performed. FIG. 13 shows a reconstructed image of the heart when the diastole of the heart is synchronized in the same ROI as in FIG. 12. FIG. 14 shows a reconstructed image of the heart when the systole of the heart is synchronized in the same ROI as in FIG. 12. In the reconstructed image before correction, for example, the boundary between the heart and the space cannot be clearly recognized at the part surrounded by a frame in FIG. 12, and the contours of the heart are blurred, resulting in a blurred reconstructed image. On the other hand, as shown in FIG. 13 and FIG. 14, in the reconstructed image after correction, it can be seen that the boundary between the heart and the space is relatively easy to be recognized in the same part as in FIG. 12.

According to the present disclosure, it is possible to reconstruct an image, or to assist in the reconstruction processing, without assuming a certain periodicity in the movement of a subject.

### [Other]

The program is a program that allows one or more computers to execute each functional unit (step). In addition, the program may be an information processing method executed by the information processing system 1 (or the processor 21 of the information processing apparatus 2).

In the embodiment, the lag value is described as being input by the user, but in a modified example, an optimal value may be automatically calculated and used. That is, the spectral decomposition unit 215 calculates the period of the frequency at the maximum peak by applying frequency analysis to the waveform data. The spectral decomposition unit 215 acquires a value of the number of data pieces that includes at least the calculated period as the setting of the lag value. According to such an aspect, more appropriate decomposed waveform data can be acquired from the projection image based on the lag value set by calculation. In addition, a reasonable lag value can be set by calculation, and this saves the user from searching for the lag value.

In the embodiment, the subject of the projection image has been described as being the lungs or heart of a living organism. However, in the modified example, the subject may include other moving objects. For example, in the modified example, the subject of the projection image may include parts of a living organism, such as the head and abdomen of a living organism, artificial lungs and hearts, or industrial objects such as machine parts in operation (e.g., testing the operation of machine parts such as gears, bearings, etc.), and fluid (e.g., observing fluid dynamics in a container, etc.).

In the embodiment, the predetermined analytical method is singular spectrum analysis, and an example of applying singular value decomposition to a Hankel matrix has been described as singular spectrum analysis. In the modified example, any method capable of acquiring decomposed waveform data from

waveform data may be used as the predetermined analytical method. Further, singular spectrum analysis using Toeplitz matrix or other methods may be used.

The present disclosure may be applied under conditions in which a beat cycle of the living organism changes during the measurement. For example, when measuring a living organism on a heated bed, the present disclosure may be applied under measurement conditions such as the timing at which anesthesia starts to take effect on the living organism, the timing at which anesthesia starts to wear off on the living organism, etc.

Furthermore, the present disclosure may be provided in each of the following aspects.
(1) An information processing system, comprising: circuitry configured to: acquire waveform data, the waveform data being indicated as feature values obtained from each of the plurality of projection images taken by a CT device; acquire decomposed waveform data by applying a predetermined analytical method to the waveform data, the decomposed waveform data being data of a waveform obtained by decomposing the waveform data; and determine a projection image to be used for reconstruction from among the plurality of projection images based on the waveform indicated by the decomposed waveform data.

According to such an aspect, even when elements such as noise and oscillation occur, a clearer image can be reconstructed from the projection image.
(2) The information processing system according to (1), wherein the analytical method is singular spectrum analysis, the circuitry is further configured to: receive a setting of a lag value and singular value selection information, the lag value is a value for specifying a matrix of the singular spectrum analysis from the feature values, the singular value selection information indicates which singular value is to be selected from among a plurality of singular values acquired from the matrix, and apply the singular spectrum analysis to the waveform data based on the lag value and acquire the decomposed waveform data corresponding to a singular value selected by the singular value selection information.

According to such an aspect, more appropriate decomposed waveform data can be acquired from the projection image based on the set lag value and singular value.
(3) The information processing system according to (2), wherein the circuitry is further configured to: receive a setting of the lag value from a user on a screen on which the waveform data is visually recognizable to the user.

According to such an aspect, more appropriate decomposed waveform data can be acquired from the projection image based on a lag value arbitrarily set by the user.
(4) The information processing system according to (2), wherein the circuitry is further

configured to: calculate a period of a frequency at a maximum peak by applying frequency analysis to the waveform data, and acquire a value of a number of data pieces that includes at least a calculated period as a setting of the lag value.

According to such an aspect, more appropriate decomposed waveform data can be acquired from the projection image based on the lag value set by calculation. In addition, a reasonable lag value can be set by calculation, and this saves the user from searching for the lag value.
(5) The information processing system according to any one of (1) to (4), wherein: the analytical method is singular spectrum analysis, the decomposed waveform data includes trend waveform data and oscillatory waveform data, the trend waveform data is data of a waveform that has been decomposed as a component indicating a trend of the waveform data, the oscillatory waveform data is data of a waveform that has been decomposed as a component indicating oscillation of the waveform data, the circuitry is further configured to: acquire the trend waveform data from a maximum singular value corresponding to a largest singular value among singular values obtained by the singular spectrum analysis; and acquire the oscillatory waveform data from at least one or more singular values selected from singular values other than the maximum singular value among singular values obtained by the singular spectrum analysis, and the synchronization processing unit is configured to determine a projection image to be used for reconstruction based on the waveform indicated by at least one of the trend waveform data and the oscillatory waveform data.

According to such an aspect, it is possible to more appropriately determine an image to be used for reconstruction from the trend waveform data and the oscillatory waveform data obtained by singular spectrum analysis.
(6) The information processing system according to any one of (1) to (5), wherein: the plurality of projection images includes at least a part of lungs of a living organism as a subject, the decomposed waveform data includes data of a waveform as an oscillation component indicated by the waveform data, and the circuitry is further configured to: determine an exhalation area and an inhalation area from oscillation indicated by the decomposed waveform data, the exhalation area being an area where intensity of the oscillation is lower than a predetermined value, the inhalation area being an area where intensity of the oscillation is higher than the predetermined value; determine that a projection image in which the feature value becomes minimum at a part corresponding to the exhalation area in the waveform data is a projection image corresponding to exhalation of the living organism; determine that a projection image in which the feature value becomes maximum at a part corresponding to the inhalation area in the waveform data is a projection image corresponding to inhalation of the living organism; and determine a projection image to be used for reconstructing the lungs from projection images corresponding to the determined exhalation and inhalation, respectively.

According to such an aspect, it is possible to more appropriately determine a projection image to be used for reconstructing the lungs from oscillation components indicated by the decomposed waveform data.
(7) The information processing system according to (6), wherein: the plurality of projection images includes at least a part of a heart of the living organism as a subject, the circuitry is further configured to: determine that a projection image in which a feature value becomes a local maximum value at a part corresponding to the exhalation area in the waveform data is a projection image corresponding to a systole of the heart; determine that a projection image in which a feature value becomes a local minimum value at a part corresponding to the exhalation area in the waveform data is a projection image corresponding to a diastole of the heart; and determine a projection image to be used for reconstructing the heart from projection images corresponding to the determined systole and diastole, respectively.

According to such an aspect, it is possible to more appropriately determine a projection image to be used for reconstructing the heart from oscillation components indicated by the decomposed waveform data.
(8) An information processing system, comprising: circuitry configured to: acquire waveform data, the waveform data being indicated as feature values obtained from each of the plurality of projection images taken by a CT device; and allow a screen including a waveform display area and a condition reception area to be displayed, the waveform display area being an area in which the waveform data and decomposed waveform data are superimposed and displayed, the decomposed waveform data being data of a waveform obtained by applying singular spectrum analysis to the waveform data, the condition reception area being an area for receiving input of a setting related to singular spectrum analysis for the waveform data.

According to such an aspect, it is possible to receive input for setting the conditions of the singular spectrum analysis while the user visually recognizes the waveform data and the decomposed waveform data.
(9) The information processing system according to (8), wherein: the screen further displays a projection image display area that is an area where the acquired projection image is displayed, and the condition reception area is configured to receive a setting of a range in which the waveform data is acquired from the projection image.

According to such an aspect, it is possible to receive input for setting the conditions of the projection image to which the singular spectrum analysis is applied while the user visually recognizes the projection image.
(10) An information processing apparatus for processing a plurality of projection images taken by a CT device, comprising: a feature value acquisition unit implemented by a processor configured to acquire waveform data, the waveform data being indicated as feature values obtained from each of the plurality of projection images; a spectral decomposition unit implemented by a processor configured to acquire decomposed waveform data by applying a predetermined analytical method to the waveform data, the decomposed waveform data being data of a waveform obtained by decomposing the waveform data; and a synchronization processing unit implemented by a processor configured to determine a projection image to be used for reconstruction from among the plurality of projection images based on the waveform indicated by the decomposed waveform data.
(11) An information processing method comprising: acquiring waveform data to be indicated as feature values obtained from each of the plurality of projection images taken by a CT device; acquiring decomposed waveform data by applying a predetermined analytical method to the waveform data, the decomposed waveform data being data of a waveform obtained by decomposing the waveform data; and determining a projection image to be used for reconstruction from among the plurality of projection images based on the waveform indicated by the decomposed waveform data.
(12) A non-transitory computer-readable storage medium storing a program configured to allow a computer to function as the at least one or more processors of the information processing system according to any one of (1) to (9).

Of course, the present disclosure is not limited thereto.

Finally, various embodiments of the present disclosure have been described, but these are presented as examples and are not intended at all to limit the scope of the disclosure. Novel embodiments can be implemented in various other forms, and various omissions, replacements, and modifications can be made within the scope of the spirit of the disclosure. The embodiments and their modifications are included in the scope and the spirit of the disclosure and are included in the scope of the disclosure described in claims and the equivalent scope thereof.

What is claimed is:

1. An information processing system, comprising:
circuitry configured to:
acquire waveform data, the waveform data being indicated as feature values obtained from each of the plurality of projection images taken by a CT device;
acquire decomposed waveform data by applying a predetermined analytical method to the waveform data, the decomposed waveform data being data of a waveform obtained by decomposing the waveform data; and
determine a projection image to be used for reconstruction from among the plurality of projection images based on the waveform indicated by the decomposed waveform data.

2. The information processing system according to claim 1,
wherein the analytical method is singular spectrum analysis,
the circuitry is further configured to:
receive a setting of a lag value and singular value selection information, the lag value is a value for specifying a matrix of the singular spectrum analysis from the feature values, the singular value selection information indicates which singular value is to be selected from among a plurality of singular values acquired from the matrix, and
apply the singular spectrum analysis to the waveform data based on the lag value and acquire the decomposed waveform data corresponding to a singular value selected by the singular value selection information.

3. The information processing system according to claim 2,
wherein the circuitry is further configured to:
receive a setting of the lag value from a user on a screen on which the waveform data is visually recognizable to the user.

4. The information processing system according to claim 2,
wherein the circuitry is further configured to:
calculate a period of a frequency at a maximum peak by applying frequency analysis to the waveform data, and
acquire a value of a number of data pieces that includes at least a calculated period as a setting of the lag value.

5. The information processing system according to claim 1, wherein:
the analytical method is singular spectrum analysis,
the decomposed waveform data includes trend waveform data and oscillatory waveform data,
the trend waveform data is data of a waveform that has been decomposed as a component indicating a trend of the waveform data,
the oscillatory waveform data is data of a waveform that has been decomposed as a component indicating oscillation of the waveform data,
the circuitry is further configured to:
acquire the trend waveform data from a maximum singular value corresponding to a largest singular value among singular values obtained by the singular spectrum analysis;
acquire the oscillatory waveform data from at least one or more singular values selected from singular values other than the maximum singular value among singular values obtained by the singular spectrum analysis; and
determine a projection image to be used for reconstruction based on the waveform indicated by at least one of the trend waveform data and the oscillatory waveform data.

6. The information processing system according to claim 1, wherein:
the plurality of projection images includes at least a part of lungs of a living organism as a subject,
the decomposed waveform data includes data of a waveform as an oscillation component indicated by the waveform data, and
the circuitry is further configured to:
determine an exhalation area and an inhalation area from oscillation indicated by the decomposed waveform data, the exhalation area being an area where intensity of the oscillation is lower than a predetermined value, the inhalation area being an area where intensity of the oscillation is higher than the predetermined value;
determine that a projection image in which the feature value becomes minimum at a part corresponding to the exhalation area in the waveform data is a projection image corresponding to exhalation of the living organism;
determine that a projection image in which the feature value becomes maximum at a part corresponding to the inhalation area in the waveform data is a projection image corresponding to inhalation of the living organism; and
determine a projection image to be used for reconstructing the lungs from projection images corresponding to the determined exhalation and inhalation, respectively.

7. The information processing system according to claim 6, wherein:
the plurality of projection images includes at least a part of a heart of the living organism as a subject,
the circuitry is further configured to:
determine that a projection image in which a feature value becomes a local maximum value at a part corresponding to the exhalation area in the waveform data is a projection image corresponding to a systole of the heart;
determine that a projection image in which a feature value becomes a local minimum value at a part corresponding to the exhalation area in the waveform data is a projection image corresponding to a diastole of the heart; and
determine a projection image to be used for reconstructing the heart from projection images corresponding to the determined systole and diastole, respectively.

8. An information processing system, comprising:
circuitry configured to:
acquire waveform data, the waveform data being indicated as feature values obtained from each of the plurality of projection images taken by a CT device; and
allow a screen including a waveform display area and a condition reception area to be displayed,
the waveform display area being an area in which the waveform data and decomposed waveform data are superimposed and displayed, the decomposed waveform data being data of a waveform obtained by applying singular spectrum analysis to the waveform data,
the condition reception area being an area for receiving input of a setting related to singular spectrum analysis for the waveform data.

9. The information processing system according to claim 8, wherein:
the screen further displays a projection image display area that is an area where the acquired projection image is displayed, and
the condition reception area is configured to receive a setting of a range in which the waveform data is acquired from the projection image.

10. An information processing method comprising:
acquiring waveform data to be indicated as feature values obtained from each of the plurality of projection images taken by a CT device;
acquiring decomposed waveform data by applying a predetermined analytical method to the waveform data, the decomposed waveform data being data of a waveform obtained by decomposing the waveform data; and
determining a projection image to be used for reconstruction from among the plurality of projection images based on the waveform indicated by the decomposed waveform data.

11. A non-transitory computer-readable storage medium storing a program configured to allow a computer to function as the at least one or more processors of the information processing system according to claim 1.
